(19) United States
(12) Patent Application Publication
(10) Pub. No.: US 2022/0235111 A1
(43) Pub. Date: Jul. 28, 2022

(54) Variant Single-Chain Insulin Analogues

(71) Applicant: CASE WESTERN RESERVE UNIVERSITY, Cleveland, OH (US)

(72) Inventors: Michael A. WEISS, Indianapolis, IN (US); Michael D. GLIDDEN, II, Cleveland, OH (US); Faramarz ISMAIL-BEIGI, Cleveland, OH (US)

(21) Appl. No.: 17/612,047

(22) PCT Filed: May 18, 2020
(86) PCT No.: PCT/US20/33493

§ 371 Date: Nov. 17, 2021

Related U.S. Application Data
(60) Provisional application No. 62/849,363, filed on May 17, 2019.

(51) Int. Cl. C07K 14/62; A61P 3/10

(52) U.S. Cl. CPC C07K 14/62; A61K 38/00; A61P 3/10

(57) ABSTRACT

A single-chain insulin analogue containing (i) diverse amino-acid substitutions at position A14; (ii) wild-type or variant residues at positions A8 and A14; and (ii) an engineered C-domain segment of lengths 4-6 containing a specific set of Alanine substitutions and/or deletions derived from the prototype C-domain sequence Glu-Glu-Gly-Pro-Arg-Arg. The analogue may otherwise be an analogue of a mammalian insulin, such as human insulin, may optionally include standard or non-standard modifications that (i) augment the stability of insulin, (ii) cause a shift in the isoelectric point to enhance or impair the solubility of the protein at neutral pH or (iii) reduce cross-binding of the protein to the Type I IGF receptor. Formulations of the above analogues at successive strengths U-100 to U-1000 in soluble solutions under acidic or neutral pH values (e.g., pH 3.0-4.2 and 6.5-7.8, respectively) and optionally in the presence of zinc ions at a molar ratio of 2.2-10 zinc ions per six insulin analogue monomers. A method of treating a patient with diabetes mellitus comprising the administration of a physiologically effective amount of the protein or a physiologically acceptable salt thereof to a patient. Use of a single-chain insulin analogue of the present invention in an insulin delivery device (such as a pump or pen) is envisioned.

### CROSS REFERENCE TO RELATED APPLICATIONS

This application claims benefit of pending U.S. Provisional Application No. 62/849,363 filed on May 17, 2019, the contents of which are incorporated by reference herein.

### STATEMENT REGARDING FEDERALLY SPONSORED RESEARCH OR DEVELOPMENT

This invention was made with government support under grant numbers DK040949 and DK074176 awarded by the National Institutes of Health. The government has certain rights in the invention.

### BACKGROUND OF THE INVENTION

This invention relates to polypeptide hormone analogues that exhibits enhanced pharmaceutical properties, such as increased increased thermodynamic stability, augmented resistance to thermal fibrillation above room temperature, decreased mitogenicity, and/or altered pharmacokinetic and pharmacodynamic properties, i.e., conferring more prolonged duration of action or more rapid duration of action relative to soluble formulations of the corresponding wild-type human hormone. More particularly, this invention relates to insulin analogues consisting of a single polypeptide chain that contains a novel class of foreshortened connecting (C) domains between A and B domains. Of length 4-11 residues, the C domains of this class consist of an N-terminal acidic element and a C-terminal segment containing at least one basic amino-acid residue. The single-chain insulin analogues of the present invention exhibit variation in pharmacodynamic (PD) properties depending on the identities of the amino-acid residues at positions A8 and/or A14. Such analogues may optionally contain standard or non-standard amino-acid substitutions at other sites in the A or B domains.

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—often confer multiple biological activities. A benefit of non-standard proteins would be to achieve augmented resistance to degradation at or above room temperature, facilitating transport, distribution, and use. An example of a therapeutic protein is provided by insulin. Wild-type human insulin and insulin molecules encoded in the genomes of other mammals bind to insulin receptors is multiple organs and diverse types of cells, irrespective of the receptor isoform generated by alternative modes of RNA splicing or by alternative patterns of post-translational glycosylation. Wild-type insulin also binds with lower affinity to the homologous Type 1 insulin-like growth factor receptor (IGF-1R).

An example of a further medical benefit would be optimization of the stability of a protein toward unfolding or degradation. Such a societal benefit would be enhanced by the engineering of proteins more refractory than standard proteins with respect to degradation at or above room temperature for use in regions of the developing world where electricity and refrigeration are not consistently available. Analogues of insulin consisting of a single polypeptide chain and optionally containing non-standard amino-acid substitutions may exhibit superior properties with respect to resistance to thermal degradation or decreased mitogenicity. The challenge posed by its physical degradation is deepened by the pending epidemic of diabetes mellitus in Africa and Asia. Because fibrillation poses the major route of degradation above room temperature, the design of fibrillation-resistant formulations may enhance the safety and efficacy of insulin replacement therapy in such challenged regions.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinapathy, blindness, and renal failure.

Insulin is a small globular protein that plays a central role in metabolism in vertebrates. Insulin contains two chains, an A chain, containing 21 residues, and a B chain containing 30 residues. The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B chain (residue B30) to the N-terminal residue of the A chain (FIG. 1A). A variety of evidence indicates that it consists of an insulin-like core and disordered connecting peptide (FIG. 1B). Formation of three specific disulfide bridges (A6-A11, A7-B7, and A20-B19; FIGS. 1A and 1B) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM). The sequence of insulin is shown in schematic form in FIG. 1C. Individual residues are indicated by the identity of the amino acid (typically using a standard one-letter code), the chain and sequence position. Pertinent to the present invention is the invention of novel foreshortened C domains of length 4-11 residues in place of the 36-residue wild-type C domain characteristic of human proinsulin.

Fibrillation, which is a serious concern in the manufacture, storage and use of insulin and insulin analogues for the treatment of diabetes mellitus, is enhanced with higher temperature, lower pH, agitation, or the presence of urea, guanidine, ethanol co-solvent, or hydrophobic surfaces. Current US drug regulations demand that insulin be discarded if fibrillation occurs at a level of one percent or more. Because fibrillation is enhanced at higher temperatures, patients with diabetes mellitus optimally must keep insulin refrigerated prior to use. Fibrillation of insulin or an insulin analogue can be a particular concern for such patients utilizing an external insulin pump, in which small amounts of insulin or insulin analogue are injected into the patient's body at regular intervals. In such a usage, the insulin or insulin analogue is not kept refrigerated within the pump apparatus, and fibrillation of insulin can result in blockage of the catheter used to inject insulin or insulin analogue into the body, potentially resulting in unpredictable fluctuations in blood glucose levels or even dangerous hyperglycemia. Insulin exhibits an increase in degradation rate of 10-fold or more for each 10° C. increment in temperature above 25° C.; accordingly, guidelines call for storage at temperatures <30° C. and preferably with refrigeration. Fibrillation of basal insulin analogues formulated as soluble solutions at pH less than 5 (such as Lantus® (Sanofi-Aventis), which contains an unbuffered solution of insulin glargine and zinc ions at pH 4.0) also can limit their self lives due to physical degradation at or above room temperature; the acidic conditions employed in such formulations impairs insulin self-assembly and weakens the binding of zinc ions, reducing the extent to which the insulin analogues can be protected by sequestration within zinc-protein assemblies.

Insulin is susceptible to chemical degradation, involving the breakage of chemical bonds with loss of rearrangement of atoms within the molecule or the formation of chemical bonds between different insulin molecules. Such changes in chemical bonds are ordinarily mediated in the unfolded state of the protein, and so modifications of insulin that augment its thermodynamic stability also are likely to delay or prevent chemical degradation. Insulin is also susceptible to physical degradation. The present theory of protein fibrillation posits that the mechanism of fibrillation proceeds via a partially folded intermediate state, which in turn aggregates to form an amyloidogenic nucleus. In this theory, it is possible that amino-acid substitutions that stabilize the native state may or may not stabilize the partially folded intermediate state and may or may not increase (or decrease) the free-energy barrier between the native state and the intermediate state. Therefore, the current theory indicates that the tendency of a given amino-acid substitution in the two-chain insulin molecule to increase or decrease the risk of fibrillation is highly unpredictable. Models of the structure of the insulin molecule envisage near-complete unfolding of the three-alpha helices (as seen in the native state) with parallel arrangements of beta-sheets formed successive stacking of B-chains and successive stacking of A-chains; native disulfide pairing between chains and within the A-chain is retained. Such parallel cross-beta sheets require substantial separation between the N-terminus of the A-chain and C-terminus of the B-chain (>30 Å), termini ordinarily in close proximity in the native state of the insulin monomer (<10 Å). Marked resistance to fibrillation of single-chain insulin analogues with foreshortened C-domains is known in the art and thought to reflect a topological incompatibility between the splayed structure of parallel cross-beta sheets in an insulin protofilament and the structure of a single-chain insulin analogue with native disulfide pairing in which the foreshortened C-domain constrains the distance between the N-terminus of the A-chain and C-terminus of the B-chain to be unfavorable in a protofilament. The three-dimensional structure of an active and ultra-stable single-chain insulin, 57 residues in length and containing C-domain Gly-Gly-Gly-Pro-Arg-Arg (GGGPRR) stands in contrast to an inactive single-chain analogue that contains a peptide bind between Lys^{B29} and Gly^{A1} (i.e., des-B30 SCI of 50 residues in length).

The present invention was motivated by the goal of harnessing the augmented stability conferred upon insulin by chemical tethers between the A and B chains (e.g., between the ε-amino group of Lys^{B29} and the α-amino group of Gly^{m}) and by foreshortened C domains. The latter analogues are designated single-chain insulin analogues (SCIs). Whereas direct peptide bonds from residues at or near the C-terminal end of the B chain (residues B28, B29 or B30) to Gly^{A1} generally result in marked impairment of biological activity, foreshortened C domains of length 4-11 provide sufficient conformational “play” to permit at least a substantial portion of receptor-binding affinity. The structural basis of such length discrimination has been suggested by the crystal structure of a “micro-receptor”/insulin complex containing a ternary complex between the hormone and two portions of the ectodomain of the insulin receptor: the N-terminal fragment L1-CR and the C-terminal fragment αCT. SCIs can in general exhibit prolonged signaling once in the bloodstream, an unfavorable pharmacodynamic property in relation to use in insulin pumps and mealtime insulin replacement therapy. It is possible that such complex and variable pharmacodynamics properties relate to molecular interactions by C-domain sequences both within the hormone analogue and with elements of the insulin receptor. Ultra-stable single-chain or two-chain insulin analogues known in the art have exhibited a puzzling aberrant prolongation of signaling as tested on intravenous bolus injection in diabetic rats. To our knowledge, no rules are known that can predict the pharmacodynamic effects of mixed-sequence C-domains (such as Glu-Glu-Gly-Pro-Arg-Arg (EEGPRR)); the latter confers biphasic pharmacodynamics properties in one B-domain context but not another.

It would be desirable, therefore, to invent single-chain insulin analogues containing a combination of amino-acid types at positions A8 and A14 in the context of a specific set of C-domain sequences such that their biological and biophysical properties would be readily optimized for favorable therapeutic applications. Examples of such applications are provided by (i) rapid action in performance in external or internal insulin pumps and (ii) biphasic action in ultra-stable and mono-component soluble formulations for use in challenged regions of the developing world. There would be a particular need for SCI sequences in which the SCI's rapid action on subcutaneous injection in a diabetic mammal would be maintained with dual “tuning”: (a) PD tuning of the tail of insulin action, whether increased or decreased such that glycemic control could be optimized in a patient with diabetes mellitus and (b) thermodynamic tuning of the protein structure such that shelf life or reservoir life could be optimized at or above room temperature.

### SUMMARY OF THE INVENTION

It is, therefore, an aspect of the present invention to provide single-chain insulin analogues that contain a specific set of C-domain sequences with or without amino-acid substitutions at positions A8 and A14. It is an additional aspect of the present invention that the thermodynamic stability of the single-chain insulin analogues may be modulated by diverse amino-acid substitutions at position A8 or A14. It is a further aspect of the present invention that absolute in vitro affinities of the single-chain insulin analogue for IR-A and IR-B are in the range 5-150% relative to wild-type human insulin and so unlikely to exhibit significantly prolonged residence times in the hormone-receptor complex. It is yet another aspect of the present invention that such optimized analogues should bind more weakly to the mitogenic IGF-1R receptor than does wild-type human insulin and indeed exhibit reduced mitogenicity in mammalian cell culture. The present invention addresses the utility of single-chain insulin analogues whose simplified C-domain sequences facilitate co-optimization of biophysical, biological and pharmacodynamic features that are favorable for therapeutic applications.

### BRIEF DESCRIPTION OF THE SEVERAL VIEWS OF THE DRAWINGS

FIG. 1A is a schematic representation of the sequence of human proinsulin including the A- and B-chains and the connecting region shown with flanking dibasic cleavage sites (filled circles) and C-peptide (open circles).

FIG. 1B is a structural model of proinsulin, consisting of an insulin-like moiety and a disordered connecting peptide (dashed line).

FIG. 1C is a schematic representation of the sequence of human insulin indicating the position of residues B27 and B30 in the B-chain.

FIG. 2A is a graph of the molar ellipticity CD spectra (per molecule) acquired at 25° C. for several examples of the present invention and insulin lispro (KP). The analogues shown are those that have the substitutions Glu^{A14} and His^{A8} (EA14+HA8), Tyr^{A14} and His^{A8} (YA14+HA8), Glu^{A14} and Thr^{A8} (EA14+TA8), Tyr^{A14} and Thr^{A8} (EA14+TA8). Insulin lispro (KP, SEQ ID NOS: 2 and 11) is provided as a control.

FIG. 2B is a graph of helix-sensitive wavelengths 222(±1) nm) reported as <[θ]_{222±1 nm}> versus temperature for several examples of the present invention and insulin lispro (KP, SEQ ID NOS 2 and 11). Samples are thermally scanned in the forward (4° C.→88° C.) and then in the reverse (88° C.→4° C.) direction. The analogues shown are those that have the substitutions Glu^{A14} and His^{A8} (EA14+HA8), Tyr^{A14} and His^{A8} (YA14+HA8), Glu^{A14} and Thr^{A8} (EA14+TA8), Tyr^{A14} and Thr^{A8} (EA14+TA8). Insulin lispro (KP) is provided as a control.

FIG. 2C is a graph of CD-monitored guanidine-induced denaturation studies (solid lines are fits) showing the percent change in <[θ]_{222±1 nm}> versus guanidine hydrochloride concentration for examples of the present invention and insulin lispro (KP). The analogues shown are those that have the substitutions Glu^{A14} and His^{A8} (EA14+HA8), Tyr^{A14} and His^{A8} (YA14+HA8), Glu^{A14} and Thr^{A8} (EA14+TA8), Tyr^{A14} and Thr^{A8} (EA14+TA8). Insulin lispro (KP, SEQ ID NOS 2 and 11) is provided as a control.

FIG. 2D is a bar graph of Gibbs energy of unfolding (AG; kcal/mol) obtained from the titration fits in FIG. 2C for examples of the present invention and insulin lispro (KP). The analogues shown are those that have the substitutions Glu^{A14} and His^{A8} (EA14+HA8), Tyr^{A14} and His^{A8} (YA14+HA8), Glu^{A14} and Thr^{A8} (EA14+TA8), Tyr^{A14} and Thr^{A8} (EA14+TA8). Insulin lispro (KP, SEQ ID NOS 2 and 11) is provided as a control.

FIG. 3A shows the glucose levels expressed as blood glucose concentration (in mg/dL; left panel) and as fraction of initial blood glucose (right panel) over time measured after subcutaneous injection of the stated analogs at a dose of 2.6 nmol/300 g rat. 50 μg KP data was obtained from a 9.7 nmol/300 g rat dose of the analog. Sample sizes were: KP 15 μg (N=10), KP 50 μg (N=11), EA14+HA8 (N=10), YA14+HA8 (N=10), EA14+TA8 (N=6), YA14+TA8 (N=12).

FIG. 3B depicts AUCs for the first 60 mins (early phase; left panel) and 60-420 mins (late phase; right panel) presented as hybrid-box-and-whisker plots yielding: AUC for individual rats (black points), the median AUC (thick black horizontal lines), and the standard error of the mean (gray vertical lines). Black vertical bars indicate maximum and minimum AUC minus outliers. Black boxes give the upper (top) and lower (bottom) quartiles. Pairwise comparisons were made for each dataset relative to the KP (insulin lispro, SEQ ID NOS: 2 and 11) at the 15 μg (2.6 nmol/300 g rat) dose: *, p<0.1; NS, not significant.

FIG. 4A shows the glucose levels expressed as blood glucose concentration (in mg/dL; left panel) and as fraction of initial blood glucose (right panel) over time measured after subcutaneous injection of the stated C domain variants showing no significant difference in PD relative to template C domain EEGPRR at a dose of 2.6 nmol/300 g rat. Sample sizes are as stated in the plot legends.

FIG. 4B shows the glucose levels expressed as blood glucose concentration (in mg/dL; left panel) and as fraction of initial blood glucose (right panel) over time measured after subcutaneous injection of unique C domain variants that exhibited profiles similar to that of KP (insulin lispro, SEQ ID NOS: 2 and 11). All analogs were injected at a dose of 2.6 nmol/300 g rat. Sample sizes are as stated in the plot legends.

FIG. 5A is a Western blot showing relative levels of total Akt kinase (Akt) and insulin receptor (IR) in the MCF-7 human breast cancer cell line in response to stimulation with examples of the insulin analogue of the claimed invention. The insulin analogues as labelled had the following sequences: 3303—SEQ ID NO: 4; 3401—SEQ ID NO: 8; 3402—SEQ ID NO: 9; 3403—SEQ ID NO:10. Insulin lispro control (KP)—SEQ ID NOS: 2 and 11.

FIG. 5B is a Western blot showing relative levels of phospho-Akt kinase in the MCF-7 human breast cancer cell line in response to stimulation with examples of the insulin analogue of the claimed invention. The insulin analogues as labelled had the following sequences: 3303—SEQ ID NO: 4; 3401—SEQ ID NO: 8; 3402—SEQ ID NO: 9; 3403—SEQ ID NO:10. Insulin lispro control (KP)—SEQ ID NOS: 2 and 11.

FIG. 6 is a Western blot showing relative levels of Akt kinase (total protein) in the MCF-7 human breast cancer cell line in response to stimulation with examples of the insulin analogue of the claimed invention. The insulin analogues as labelled had the following sequences: 3303—SEQ ID NO: 4; 3401—SEQ ID NO: 8; 3402—SEQ ID NO: 9; 3403—SEQ ID NO:10. Insulin lispro control (KP)—SEQ ID NOS: 2 and 11.

FIG. 7 is a Western blot showing relative levels of transcription factors Fox03 and Fox01a, and the glycogen synthase kinase (GSK) and phospho-GSK kinases in response to stimulation with examples of the insulin analogue of the claimed invention. The insulin analogues as labelled had the following sequences: 3303—SEQ ID NO: 4; 3401—SEQ ID NO: 8; 3402—SEQ ID NO: 9; 3403—SEQ ID NO:10. Insulin lispro control (KP)—SEQ ID NOS: 2 and 11.

FIG. 8 is a Western blot showing relative levels of phospho-Akt kinase (P-Akt) and phospholyated insulin receptor (P-IR) in the rat HepG2 hepatoma-derived liver cell line in response to stimulation with examples of the insulin analogue of the claimed invention. The insulin analogues as labelled had the following sequences: 3303—SEQ ID NO: 4; 3401—SEQ ID NO: 8; 3402—SEQ ID NO: 9; 3403—SEQ ID NO:10. Insulin lispro control (KP)—SEQ ID NOS: 2 and 11.

FIG. 9 is a Western blot showing relative levels of phospho-Akt kinase (P-Akt) and phospholyated insulin receptor (P-IR) in the L6 rat myoblast cell line in response to stimulation with examples of the insulin analogue of the claimed invention. The insulin analogues as labelled had the following sequences: 3303—SEQ ID NO: 4; 3401—SEQ ID NO: 8; 3402—SEQ ID NO: 9; 3403—SEQ ID NO:10. Insulin lispro control (KP)—SEQ ID NOS: 2 and 11.

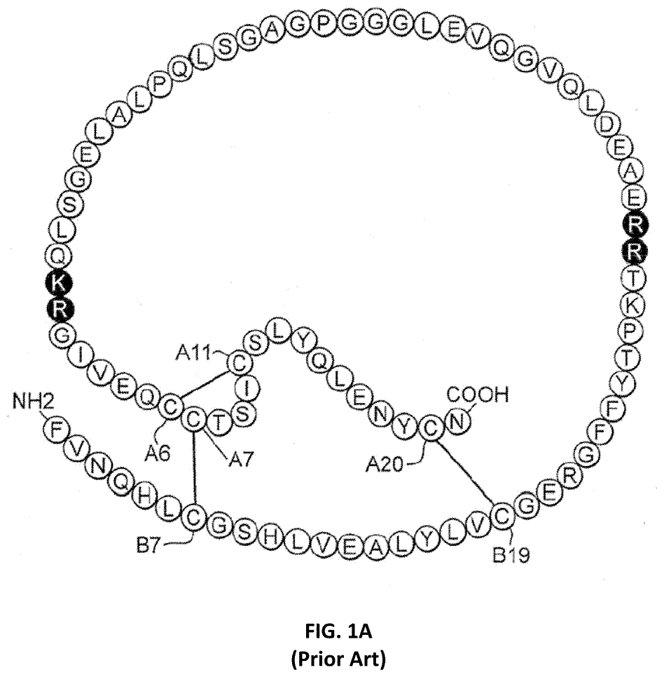

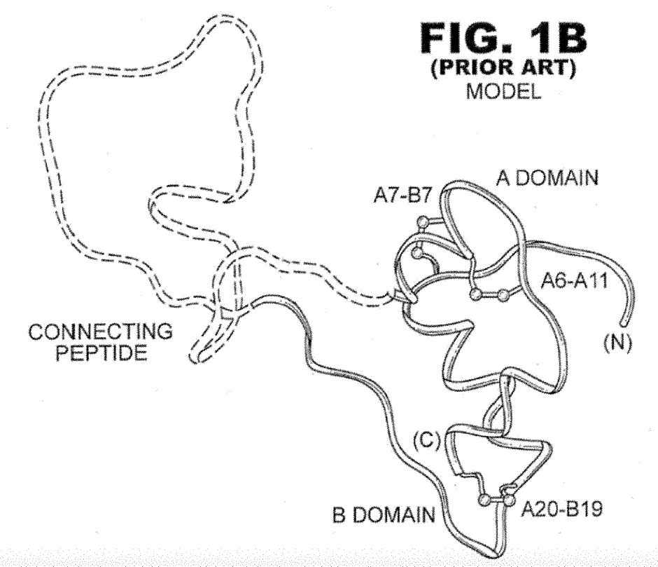

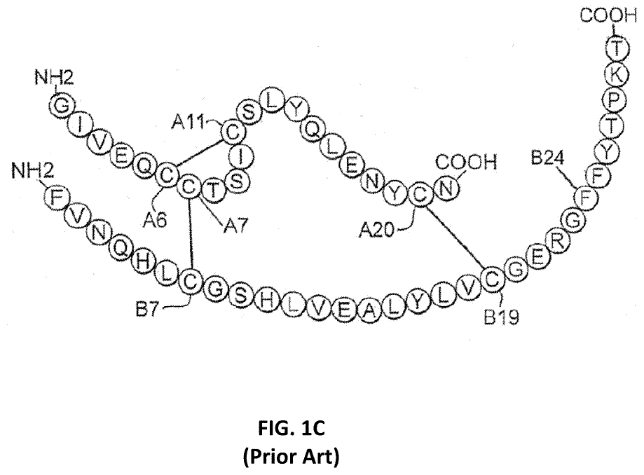

### DETAILED DESCRIPTION OF THE INVENTION

The present invention is directed toward a single-chain insulin analogue that provides (i) enhanced stability, solubility and resistance to fibrillation due to the presence of a foreshortened C domain (length 4-11 residues) and (ii) ready and convenient co-optimization of biological, biophysical and pharmacodynamics properties. The single-chain insulin analogues of the present invention may have an isoelectric point between 4.0 and 6.0 (and so be suitable for formulation under neutral pH conditions as a rapid-acting insulin analogue formulation) or may have an isoelectric point between 6.5 and 8.0 (and so be suitable for formulation under acidic pH conditions as a basal insulin analogue formulation). Molecular embodiments of this strategy were prepared by biosynthetic expression in the yeast Pichia pastoris. The parent SCI has previously been disclosed in U.S. Pat. No. 9,499,600, issued Nov. 22, 2016, which is incorporated by reference herein. The variant SCIs of the present invention differ from this prototype in containing amino-acid substitutions at positions A8 and/or A14 (Table 1A, B). The foreshortened C domains of the present invention are given in Table 1C. The N-terminal residue of the C domain is acidic (Aspartic Acid or Glutamic Acid) in order to impair binding of the analogues to the mitogenic Type 1 IGF receptor (IGF-1R) relative to insulin receptor isoforms (IR-A and IR-B).

TABLE 1
Sequence features of SCI variant and control insulin analogs
A. Control Analogs

| Analog | Mutations Relative to |
|---|---|
| Name | WT Insulin |
| KP (lispro) | Lys^{B28}, Pro^{B29}; no C domain |
| EA14 + HA8^{a} | Asp^{B28}, Pro^{B29}, His^{A8}, Glu^{A14}; |
| | EEGPRR C domain |

^{a}EA14 + HA8 has an identical sequence to previously characterized
SCI-b (1, 2).
B. A Domain SCI Variants

| Analog | A8 and A14 |
|---|---|
| Name | Residues Present^{b} |
| EA14 +TA8 | Glu^{A14} and Thr^{A8} |
| YA14 +HA8 | Tyr^{A14} and His^{A8} |
| YA14 +TA8 | Tyr^{A14} and Thr^{A8} |

^{b}All A8 and A14 variants have the template C domain sequence
EEGPRR (1, 2).
C. C Domain SCI Variants

| Analog | Mutations relative to |
|---|---|
| Name | template SCI-b^{c} |
| EAGPRR | Ala^{C2} |
| AEGPRR | Ala^{C1} |
| EEGARR | Ala^{C4} |
| EAGARR | Ala^{C2} and Ala^{C4} |
| EFGPRR | Phe^{C2} |
| EDGPRR | Asp^{C2} |
| EAAAAA | Ala^{C2}, Ala^{C3}, Ala^{C4}, |
| | Ala^{C5}, Ala^{C6} |
| EGPRR | del(Glu^{C2}) |

^{c}All C domain variants possess Glu^{A14} and His^{A8} as in the EA14 +
HA8 (SCI-b) template.

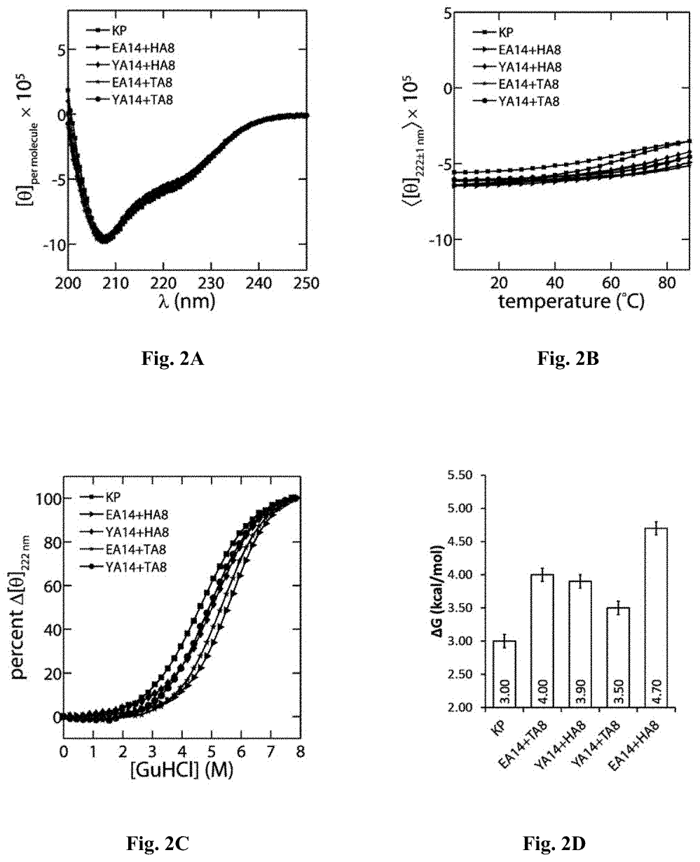

In FIG. 2 are shown biophysical data pertaining to variants of a parent prototype SCI in which the amino-acid substitutions at position A8 and/or A14 are reverted, singly or together, to the respect human residues Thr^{A8} and/or Tyr^{A14}. The analogues shown are those that have the substitutions Glu^{A14} and His^{A8} (EA14+HA8), Tyr^{A14} and His^{A8} (YA14+HA8), Glu^{A14} and Thr^{A8} (EA14+TA8), Tyr^{A14} and Thr^{A8} (EA14+TA8). Insulin lispro (KP) is provided as a control. Whereas the far-ultra-violet CD spectra of this group of SCI analogues are similar (FIG. 2A), the human residues in part attenuate the augmented stability exhibited by the SCIs relative to two-chain control analog insulin lispro.

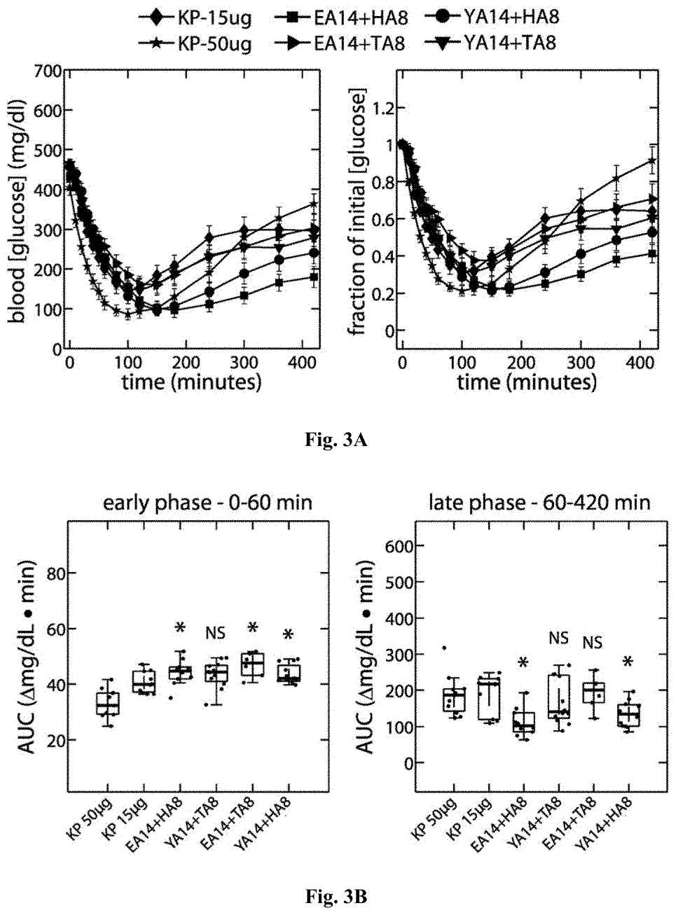

In FIG. 3 are shown the results of biological testing of the above SCI variants in male Sprague-Dawley rats rendered diabetic by beta-cell poison streptozotocin. The analogues shown are those that have the substitutions Glu^{A14} and His^{A8} (EA14+HA8), Tyr^{A14} and His^{A8} (YA14+HA8), Glu^{A14} and Thr^{A8} (EA14+TA8), Tyr^{A14} and Thr^{A8} (EA14+TA8). Insulin lispro (KP) is provided as a control at a 15 The SCIs were administered by intravenous (IV) bolus injection in relation to IV injection of insulin lispro. The data demonstrate that the relative magnitude of the tail of insulin action from 120-420 min post-injection differs among this set of analogs.

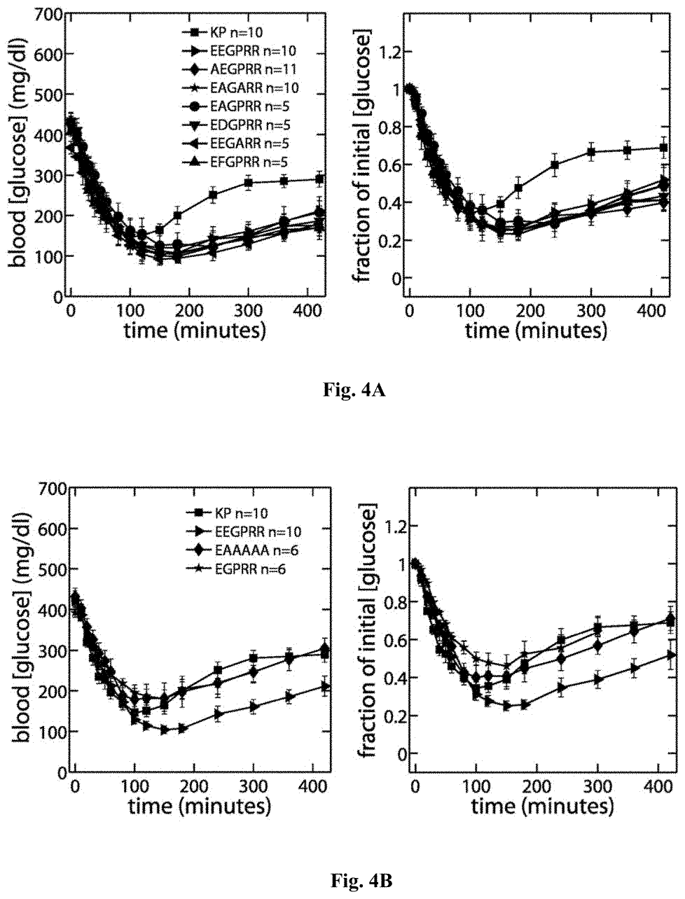

In FIG. 4 are shown similar biological data pertaining to SCIs differing in C-domain sequence of length in the presence of His^{A8} and Glu^{A14} substitutions. The biphasic PD property of the parent SCI is retained with diverse C-domain sequences or lengths, with the exception of a simplified Glu-(Ala). C domain, which is essentially “tail-less.” These data demonstrate that the relative magnitude of the tail can depend in detail on both A-domain substitutions and C-domain sequence in combination.

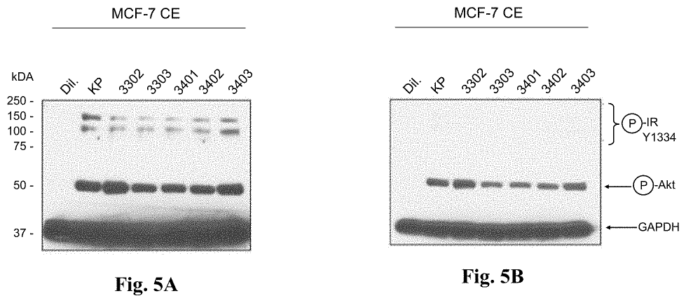

While not intending to be constrained by theory, the A8 side chain is positioned to interact through electrostatic interactions with the helical dipole axis (white horizontal arrow in FIG. 5), including as a potential N-Cap residue, and with the negative charge of Glu^{A4} via (i, i−4) side-chain interactions. Further modulation of the electrostatic features of this α-helical segment can be provided by substitutions at position A8 via (i, i+4) side-chain interactions. The wild-type residue at position A8, which is Threonine in human insulin, contains a β-branched side chain that is suboptimal with respect to both α-helical propensity and C-Cap propensity. It is an aspect of the present invention that substitutions at position A8 can alter the PD profile of an SCI.

The SCIs of the present invention may also contain substitutions at position A14 as exemplified in Table 2. Although not constrained by theory, these data suggest that substitution of the hyper-exposed Tyr^{A14} on the surface of wild-type insulin by Glu or other less hydrophobic side chains may mitigate an unfavorable “reverse-hydrophobic effect”—thereby augmenting thermodynamic stability—and simultaneously remove a potential aromatic site of chemical degradation. It is another aspect of the present invention that substitutions at position A14—and a combination of substitutions at positions A8 and A14—can alter both the thermodynamic stability and the PD profile of an SCI. In the data provided in Table 2, thermodynamic stabilities were measured by CD-monitored chemical denaturation and in selected cases also by ¹H-NMR amide proton exchange as described in the case of the prototype SCIs.

TABLE 2
Thermodynamic Stabilities of SCI Analogs.

| | ΔG_{CD} | | | ΔG_{NMR} |
|---|---|---|---|---|
| A14 Residue | (kcal/mol)^{a} | m-value | C_{mid} (M)^{b} | (kcal/mol)^{c} |
| Non-polar | | | | |
| Y | 3.9 ± 0.1 | 0.74 ± 0.02 | 5.3 ± 0.1 | 3.98 ± 0.05^{d} |
| F | 4.2 ± 0.1 | 0.81 ± 0.02 | 5.2 ± 0.1 | — |
| W | 4.2 ± 0.1 | 0.76 ± 0.02 | 5.5 ± 0.1 | — |
| V | 3.9 ± 0.1 | 0.77 ± 0.01 | 5.1 ± 0.1 | — |
| G | 4.2 ± 0.1 | 0.83 ± 0.01 | 5.0 ± 0.1 | 4.20 ± 0.10 |
| A | 4.6 ± 0.1 | 0.79 ± 0.02 | 5.8 ± 0.1 | — |
| Polar | | | | |
| N | 4.2 ± 0.1 | 0.79 ± 0.02 | 5.4 ± 0.1 | 4.20 ± 0.06 |
| Q | 4.6 ± 0.1 | 0.83 ± 0.01 | 5.6 ± 0.1 | — |
| S | 4.5 ± 0.1 | 0.82 ± 0.02 | 5.5 ± 0.1 | — |
| Charged | | | | |
| D | 4.4 ± 0.1 | 0.81 ± 0.01 | 5.4 ± 0.1 | — |
| E | 4.7 ± 0.1 | 0.82 ± 0.01 | 5.7 ± 0.1 | 4.67 ± 0.01 |
| R | 4.5 ± 0.1 | 0.79 ± 0.01 | 5.7 ± 0.1 | — |

^{a} From two-state transition fits of (D-guanidine titration performed at 25° C. and pH 7.4
^{b}C_{mid} is guanidine denaturant concentration at which 50% of the protein is in the unfolded state.
^{c}From pH 7.4 amide exchange and fitting the B18 global amide resonance, unless otherwise noted. Hyphen = ΔG_{NMR} not measured by amide exchange.
^{d}From amide exchange performed at pH 2.8 and fitting the B18 global amide reaonance.

The single-chain insulin analogues of the present invention may also contain substitutions within their respective A- and B domains. B-domain substitutions can include variants known in the art to weaken self-association and thus confer rapid absorption on subcutaneous injection; examples include AspB28 (as in Novolog®; insulin aspart), Lys^{B28}-Pro^{B29} (as in Humalog®; insulin lispro) or Asp^{B28}-Pro^{B29} (as described in Hua, Q.-X., et al. 2008). The analogues of the present invention exclude the substitution His^{B10}→Asp, which has been associated with enhanced mitogenicity in cell culture and carcinogenesis in rat testing (Hansen, B. F., et al. (2011)).

In view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Di-aminobutyric acid, or Di-aminopropionic acid). Lys may also be replaced by the neutral aliphatic isostere Norleucine (Nle), which may in turn be substituted by analogues containing shorter aliphatic side chains (Aminobutyric acid or Aminopropionic acid).

In the sequences described herein, nucleotides should be understood to be represented by their standard one letter abbreviations: A for adenine, G for guanine, C for cytosine, T for thymine and U for uracil. The presence of a mixture of nucleotides may also be indicated with an abbreviation as recognized in the art: N for any base (A,C,G or T/U), R for purine (G or A), Y for pyrimidine (T/U or C), M for amino (A or C), K for keto (G or T/U), S for G or C, W for A or T/U, V for nucleotides other than T (A, C, or G), D for nucleotides other than C (A, G, or T), B for nucleotides other than A (C, G, or T), and H for nucleotides other than G (A, C, or T).

Representative analogues of the present invention were purified from an engineered strain of the yeast Pichia pastoris as described (Glidden, M. D., et al., 2018a and 2018b). The prototype SCI contained four substitutions in the insulin moiety: Thr^{A8}→His and Tyr^{A14}—Glu and with B-domain substitutions Pro^{B28}→Asp and Lys^{B29}→Glu. The respective rationales for these substitutions were as follows. His^{A8} was introduced to augment receptor-binding affinity and thermodynamic stability; Glu^{A14} was introduced to enhance stability and reduce the isoelectric point (otherwise increased by the partial charge of His^{A8}); and the Asp^{B28}→Pro^{B29} element was introduced to weaken dimerization and further reduce the isoelectric point. The pharmacodynamics features of this analogue were tested following subcutaneous injection in a rat model of diabetes mellitus as described (Menting, J. G., et al., 2014). Its biological activity, onset of action, and duration of action were defined relative insulin lispro and two single-chain analogues containing the more complex C domain previously known in the art (Glu-Glu-Gly-Pro-Arg-Arg; EEGPRR). The PD profile of the prototype SCI is biphasic as described in Glidden et al. (2018a). This biphasic property was retained on substitution by a variety of C-domain sequences (Table 1C). A variant was constructed in which the A8 residue was reverted to Threonine and in which the A14 residue was reverted to Glutamic Acid. Remarkably, its pharmacodynamic profile is essentially identical to that of insulin lispro with respect to onset of action, offset of action, and integrated potency (area over the curve). The absence of a prolonged tail despite the presence of His^{A8} and Glu^{A14} demonstrates an interplay between C-domain sequence and modifications in the insulin moiety.

We note that the presence or absence of a “tail” of insulin action following the immediate peak of activity (as observed following intravenous bolus injection in a mammal) may be advantageous or disadvantageous depending on the clinical context and administration device. On the one hand, the absence of a tail could enable more precise control of prandial dosing. For use in an insulin pump, for example, a “tail-less” form of an insulin analogue would be desirable to reduce the risk of late hypoglycemia and to enhance the robustness of control algorithms in closed-loop systems (i.e., in which an algorithm controls the pump delivery rate in response to the output of a continuous glucose monitor). Other features of the insulin analogue, such as enhanced stability or resistance to aggregation at high protein concentration, may be desirable in such a device. On the other hand, the presence of a tail could enhance overall glycemic control in a biphasic insulin formation, especially in patients with diabetes mellitus in underprivileged environments not suitable for advanced closed-loop technologies or in patients with long-standing Type 2 diabetes mellitus for whom approximate glucose control may be safer than tight control. In challenged regions of the developing world, for example, the absence of refrigeration and limited educational backgrounds of many patients with Type 2 diabetes mellitus create conditions in which ultra-stable biphasic single-chain insulin analogue formulations would be of particular clinical value. The present invention provides a method for the dual tuning of the magnitude of the tail of insulin action and the thermodynamic stability of the SCI as a globular protein structure. The nature of the amino-acid substitutions at A8 and A14 and the specific C-domain sequence may be chosen to adjust the isoelectric point (pI) of the protein as a whole, such that the pI is in the range 4.0-5.5 (as suitable for a neutral-pH rapid-acting or biphasic formulation) or in the range 6.5-8.0 (as suitable for an acidic basal or biphasic formulation).

The biological activity of insulin analogues of the claimed invention was investigated in tissue culture cells using MCF-7 human breast cancer cell, rat hepatoma-devived HepG2 cells and rat myoblast L6 cells. The insulin analogues tested had the following sequences:

3303—SEQ ID NO: 4

3401—SEQ ID NO: 8

3402—SEQ ID NO: 9

3403—SEQ ID NO: 10.

Relevant tissue culture cells were challenged with 50 nM insulin analogue (or lispro control) and harvested. Analysis of Insulin Receptor (IR) activation and downstream metabolic effects were determined by Western blot analysis of expressed proteins extracted from MCF-7 human breast cancer cells (which express Insulin Receptor-A (IR-A), Insulin Receptor-B (IR-B) and Insulin-like Growth Factor 1 Receptor (IGF-1R)), rat hepatoma HepG2 cells, and rat L6 cells expressing Insulin Receptor A (IRA). Equal amounts of total protein extracts (12-30 for MCF-7, HepG2, and L6 cells) were loaded in Bio-Rad Mini-Protean TGX gels (4-20%) for separation. Briefly, supernatants collected following centrifugation of total cell lysates and after BCA protein assay (Thermo Scientific) were added with Laemmli sample buffer (Bio-Rad) containing 10% βME, heated at 95° C. for 6 min, and centrifuged quickly before loading in gels as mentioned above. Gels were usually run (100V constant) with the 20 kD protein marker (Bio-Rad) at the very bottom. Separated proteins were then transferred onto 0.2 μm PVDF membrane (Bio-Rad) for overnight (˜22 h) in cold-room (4° C.) using 25-30V (constant). Following transfer, membranes depending upon the proteins to be probed by individual primary antibodies were blocked either in 5% BSA (Cell Signaling Technology) (for example, pIR) or 5-10% nonfat dry milk (Bio-Rad) (for example, pAkt) in 1× TBS-T (Tris-buffered saline with 0.1% Tween-20) for 2 h at room temp on an orbital shaker. Membranes were then incubated for overnight (˜22 h) in cold room (at 4° C.) on an orbital shaker with the following individual antibodies—anti-insulin receptor β (1:1,000), anti-phospho insulin receptor (Y1334) (Invitrogen) (1:3,000), anti-pAkt (Ser 473) (1:400), anti-Akt (pan) (1:1,000), anti-GAPDH (1:8,000), anti-FoxO (1:1,000), anti-phospho FoxO (1:1,000), anti-GSK-3 α/β (1:1,000), anti-phospho GSK-3 α/β (1:1,000), anti-p27(kip1) (1:4,000), anti-phospho p27(S10) (1:1,000), anti-phospho p27(T157) (0.5 μg/ml), and anti-phospho glycogen synthase (1:1,000). All these antibodies were purchased from Cell Signaling Technology except stated otherwise. After the primary antibody incubation, membranes were washed thoroughly at room temp with 1×TBS-T (3×10 min) and then incubated with goat anti-rabbit IgG-HRP (1:5,000) in either 5% BSA or 5-10% non-fat dry milk in 1×TBS-T for 1-2 h at room temp on an orbital shaker. Following secondary antibody incubation, membranes were thoroughly washed as mentioned above (3×10 min) and then the protein bands were detected employing chemiluminescence reagents (Signal Fire ECL—Cell Signaling Technology) and finally developed in a dark room using autoradiography films.

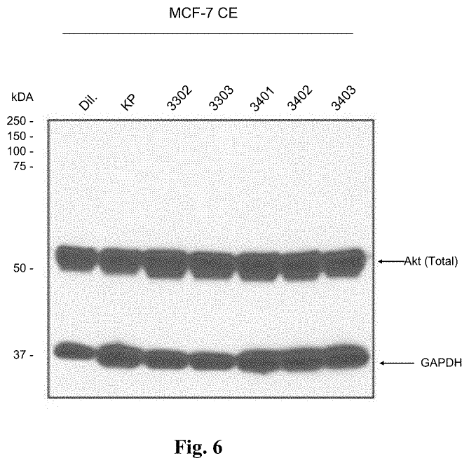

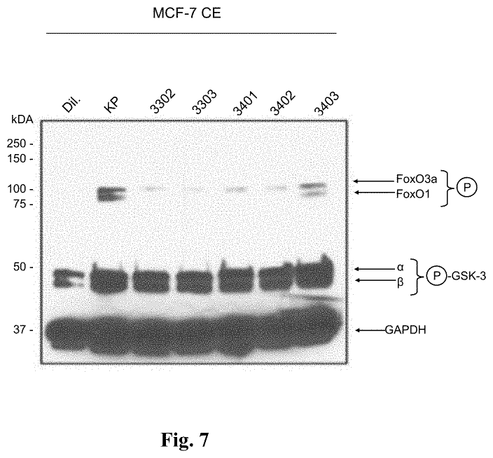

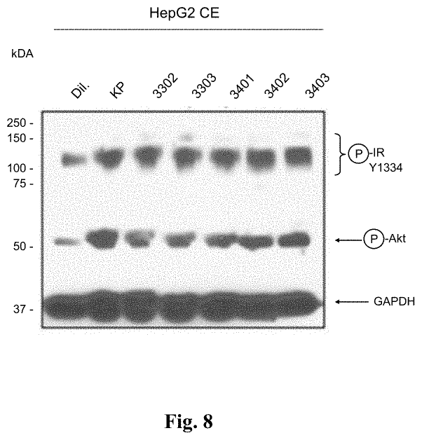

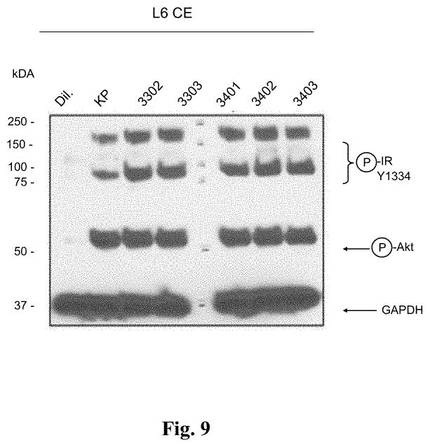

As shown in FIGS. 5A, 5B and 6, activation of IR and subsequent stimulation of Akt (also referred to as Protein kinase B) in MCF-7 cells appear to be lower when treated with 3302 & 3303 and also 3401 & 3402 relative to lispro insulin & 3403. Phosphor glycogen synthase kinase (GSK-3, both alpha and beta forms) in MCF-7 cells appear to be substantially identical between 3302, 3303, 3401 and 3402 and lispro insulin and 3403, as shown in FIG. 7. Treatments with insulin analogs without (3302 & 3303) or with one or two alanine residues (3401 & 3402, respectively) in the linker region cause noticeably lower levels of FoxO phosphorylation compared to KP insulin and 3403 having three alanines in the linker (FIG. 7). Similar results are seen for corresponding studies in HepG2 cells (FIG. 8) and L6 cells (FIG. 9). These results indicate that the analogues containing no Alanines in the C-connecting peptide/linker, or one or two alanines, may have cell cycle inhibitory effects.

The insulin analogue of the present invention may be used as a medicament, and/or for the treatment of disease, such as diabetes mellitus, or other conditions where a reduction in blood sugar levels is advisable or necessary. In some examples, the insulin analogue of the present invention may be used for the manufacture of a medicament for the treatment of diabetes mellitus.

A method for treating a patient with diabetes mellitus comprises administering a single-chain insulin analogue as described herein. It is another aspect of the present invention that the single-chain insulin analogues may be prepared either in yeast (Pichia pastoris) or subject to total chemical synthesis by native fragment ligation. The synthetic route of preparation is preferred in the case of non-standard modifications, such as D-amino-acid substitutions, halogen substitutions within the aromatic rings of Phe or Tyr, or O-linked modifications of Serine or Threonine by carbohydrates; however, it would be feasible to manufacture a subset of the single-chain analogues containing non-standard modifications by means of extended genetic-code technology or four-base codon technology (for review, see Hohsaka, T., & Sisido, M., 2012). It is yet another aspect of the present invention that use of non-standard amino-acid substitutions can augment the resistance of the single-chain insulin analogue to chemical degradation or to physical degradation. We further envision the analogues of the present invention providing a method for the treatment of diabetes mellitus or the metabolic syndrome. The route of delivery of the insulin analogue is by subcutaneous injection through the use of a syringe or pen device.

A single-chain insulin analogue of the present invention may also contain other modifications, such as a halogen atom at positions B24, B25, or B26 as described more fully in U.S. Pat. No. 8,921,313, the disclosure of which is incorporated by reference herein. An insulin analogue of the present invention may also contain a foreshortened B-chain due to deletion of residues B1-B3 as described more fully in U.S. Pat. No. 9,725,493, the disclosure of which is incorporated by reference herein.

A pharmaceutical composition may comprise such insulin analogues and which may optionally include zinc. Zinc ions may be included at varying zinc ion:protein ratios, ranging from 2.2 zinc atoms per insulin analogue hexamer to 10 zinc atoms per insulin analogue hexamer. The pH of the formulation may either be in the range pH 3.0-4.5 (as a basal formulation of a pI-shifted single-chain insulin analogue) or be in the range pH 6.5-8.0 (as a prandial insulin formulation of a single-chain insulin analogue whose pI is similar to that of wild-type insulin). In either such formulation, the concentration of the insulin analogue would typically be between about 0.6-5.0 mM; concentrations up to 5 mM may be used in vial or pen; the more concentrated formulations (U-200 or higher, including in the range U-500 through U-1000) may be of particular benefit in patients with marked insulin resistance. Excipients may include glycerol, glycine, arginine, Tris, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

The amino-acid sequence of human proinsulin is provided, for comparative purposes, as SEQ ID NO: 1.

SEQ ID NO: 1
(human proinsulin)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn

The amino-acid sequence of the A chain of human insulin is provided as SEQ ID NO: 2.

SEQ ID NO: 2
(human A chain)
Gly-Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the B chain of human insulin is provided as SEQ ID NO: 3.

SEQ ID NO: 3
(human B chain)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

The amino-acid sequence of the prototype SCI is provided as SEQ ID NO: 4.

SEQ ID NO: 4
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Glu-Glu-Gly-Pro-Arg-Arg-
Gly-Ile-Val-Glu-Gln-Cys-Cys-His-Ser-Ile-Cys-Ser-
Leu-Glu-Gln-Leu-Glu-Asn-Tyr-Cys-Asn

The amino-acid sequence of the SCIs of the present invention conform to the following as provided in SEQ ID NO: 5

The amino-acid sequence of variants of an SCI containing diverse substitutions at position A14 and/or position A8 is provided as SEQ ID NO: 5 to SEQ ID NO: 10.

SEQ ID NO: 5
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-XXa₁-XXa₂-Pro-Thr-Glu-Glu-Gly-Pro-Arg-
Arg-Gly-Ile-Val-Glu-Gln-Cys-Cys-Xaa₄-Ser-Ile-Cys-
Ser-Leu-XXa₃-Gln-Leu-Glu-Asn-Tyr-Cys-Asn

Where the XXa₁-XXa₂ element at B-domain positions B28 and B29 either (a) contains a Proline at position B28 and a non-proline residue at position B29 (as exemplified by the wild-type sequence Pro^{B28}-Lys^{B29} and its variants Pro-Arg, Pro-Glu, or Pro-Ala), (b) contains a Proline at position B29 and a non-proline at position B28 (as exemplified by Asp-Pro, Glu-Pro, Ala-Pro, Asp-Lys, Glu-Lys, and Ala-Arg) and (c) does not contain Proline at either position (as exemplified by Asp-Lys, Glu-Arg, Ala-Lys, and Ala-Arg); and where Xaa₃ may be any amino acid, and Xaa₄ is Thr, His, Ser, Glu, or Ala.

SEQ ID NO: 6
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Z-Gly-Ile-Val-Glu-Gln-Cys-
Cys-Xaa₁-Ser-Ile-Cys-Ser-Leu- Xaa₂-Gln-Leu-Glu-
Asn-Tyr-Cys-Asn

Where Xaa₁ is Thr, His, Ser, Glu or Ala; where Xaa₂ is any amino acid other than Proline; and where Z indicates a peptide of length 6 residues containing derivable from the parent sequence Glu-Glu-Gly-Pro-Arg-Arg by one, two or three Alanine substitutions (e.g., Ala-Glu-Gly-Pro-Arg-Arg, Glu-Ala-Gly-Pro-Arg-Arg, Glu-Glu-Ala-Pro-Arg-Arg, Glu-Glu-Gly-Ala-Arg-Arg, Glu-Glu-Gly-Pro-Ala-Arg, or Glu-Glu-Gly-Pro-Arg-Ala; or by two-Ala substitutions such as Glu-Ala-Gly-Pro-Arg-Ala or Glu-Glu-Ala-Ala-Arg-Arg; or by three Ala substitutions such as Glu-Ala-Gly-Ala-Arg-Ala).

SEQ ID NO: 7
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Pro-Thr-Z′-Gly-Ile-Val-Glu-Gln-
Cys-Cys-Xaa₁-Ser-Ile-Cys-Ser-Leu- Xaa₂-Gln-Leu-
Glu-Asn-Tyr-Cys-Asn

Where Xaa₁ is Thr, His, Ser, Glu or Ala; where Xaa₂ is any amino acid other than Proline; and where Z′ indicates a peptide of length 4-5 residues containing derivable from the C-domain sequences in SEQ ID NO:6 by deletion of one or two residues (e.g., Glu-Gly-Pro-Arg, Glu-Ala-Gly-Pro-Arg, Glu-Glu-Ala-Arg-Arg or Glu-Gly-Ala-Arg-Ala).

SEQ ID NO: 8
Phe Val Asn Gln His Leu Cys Gly Ser His Leu Val
Glu Ala Leu Tyr Leu Val Cys Gly Glu Arg Gly Phe
Phe Tyr Thr Asp Pro Thr Glu Glu Gly Ala Pro Arg
Arg Gly Ile Val Glu Gln Cys Cys His Ser Ile Cys
Ser Leu Glu Gln Leu Glu Asn Tyr Cys Asn
SEQ ID NO: 9
Phe Val Asn Gln His Leu Cys Gly Ser His Leu Val
Glu Ala Leu Tyr Leu Val Cys Gly Glu Arg Gly Phe
Phe Tyr Thr Asp Pro Thr Glu Glu Gly Ala Ala Pro
Arg Arg Gly Ile Val Glu Gln Cys Cys His Ser Ile
Cys Ser Leu Glu Gln Leu Glu Asn Tyr Cys Asn
SEQ ID NO: 10
Phe Val Asn Gln His Leu Cys Gly Ser His Leu Val
Glu Ala Leu Tyr Leu Val Cys Gly Glu Arg Gly Phe
Phe Tyr Thr Asp Pro Thr Glu Glu Gly Ala Ala Ala
Pro Arg Arg Gly Ile Val Glu Gln Cys Cys His Ser
Ile Cys Ser Leu Glu Gln Leu Glu Asn Tyr Cys Asn
SEQ ID NO: 11
(lispro B chain)
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr- Lys- Pro-Thr

The word “comprising” and forms of the word “comprising” as used in this description and in the claims does not limit the invention claimed to exclude any variants or additions.

Based upon the foregoing disclosure, it should now be apparent that an ultra-stable single-chain insulin analogue may be made compatible with either an unperturbed duration of insulin signaling or a “tail” of insulin action tuneable through the co-engineering of amino-acid substitutions at positions A8 and A8 in the context of a foreshortened and simplified C domain. The resulting single-chain insulin analogues provided will carry out the objects set forth hereinabove. Namely, these modified proteins exhibit enhanced resistance to fibrillation while retaining desirable pharmacokinetic and pharmacodynamic features (conferring rapid or prolonged rates of absorption from a subcutaneous depot with or without a tail of insulin action as may be therapeutically desired) and maintaining at least a fraction of the biological activity of wild-type insulin. It is, therefore, to be understood that any variations evident fall within the scope of the claimed invention and thus, the selection of specific component elements can be determined without departing from the spirit of the invention herein disclosed and described.

The following literature is cited to demonstrate that the testing and assay methods described herein would be understood by one of ordinary skill in the art.
Derewenda, U., Derewenda, Z., Dodson, E. J., Dodson, G. G., Bing, X., and Markussen, J. 1991. X-ray analysis of the single chain B29-A1 peptide-linked insulin molecule. A completely inactive analogue. J. Mol. Biol. 220: 425-433.
Doig, A. J, & Baldwin, R. L. N- and C-capping preferences for all 20 amino acids in alpha-helical peptides. 1995. Protein Sci. 4: 1325-36.
Hansen, B. F., Kurtzhals, P., Jensen, A. B., Dejgaard, A., Russell-Jones, D. 2011. Insulin X10 revisited: a super-mitogenic insulin analogue. Diabetologia. 54: 2226-31.
Glidden, M. D., Aldabbagh, K., Phillips., N. B., Carr., K., Chen, Y. S., Whittaker, J., Phillips, M., Wickramasinghe, N. P., Rege, N., Swain, M., Peng, Y., Yang, Y., Lawrence, M. C., Yee, V. C., Ismail-Beigi, F., & Weiss, M. A. 2017. An ultra-stable single-chain insulin analog resists thermal inactivation and exhibits biological signaling duration equivalent to the native protein. J. Biol. Chem. pii: jbc.M117.808626. doi: 10.1074/jbc.M117.808626 [Epub ahead of print]; in print: 293: 69-8
Glidden M D, Yang Y, Smith N A, Phillips N B, Carr K, Wickramasinghe N P, Ismail-Beigi F, Lawrence M C, Smith B J, Weiss M A. 2017. Solution structure of an ultra-stable single-chain insulin analog connects protein dynamics to a novel mechanism of receptor binding. J. Biol. Chem. pii: jbc.M117.808667. doi: 10.1074/jbc.M117.808667 [Epub ahead of print]; in print 293: 47-68.
Hohsaka, T., & Sisido, M. 2012. Incorporation of non-natural amino acids into proteins. Curr. Opin. Chem. Biol. 6: 809-15.
Hua, Q. X., Nakagawa, S. H., Jia, W., Huang, K., Phillips, N. B., Hu, S. & Weiss, M. A. (2008) Design of an active ultrastable single-chain insulin analog: synthesis, structure, and therapeutic implications. J. Biol. Chem. 283: 14703-14716.
Kristensen, C., Andersen, A. S., Hach, M., Wiberg, F. C., Schïffer, L., & Kjeldsen, T. 1995. A single-chain insulin-like growth factor I/insulin hybrid binds with high affinity to the insulin receptor. Biochem. J. 305: 981-6.
Lee, H. C., Kim, S. J., Kim, K. S., Shin, H. C., & Yoon, J. W. 2000. Remission in models of type 1 diabetes by gene therapy using a single-chain insulin analogue. Nature 408, 483-8. Retraction in: Lee H C, Kim K S, Shin H C. 2009. Nature 458: 600.
Menting, J. G., Yang, Y., Chan, S. J., Phillips, N. B., Smith, B. J., Whittaker, J., Wickramasinghe, N. P., Whittaker, L. J., Pandyarajan, V., Wan, Z. L., Yadav, S. P., Carroll, J. M., Strokes, N., Roberts, C. T., Jr, Ismail-Beigi, F., Milewski, W., Steiner, D. F., Chauhan, V. S., Ward, C. W., Weiss, M. A., & Lawrence, M. C. 2014. Protective hinge in insulin opens to enable its receptor engagement. Proc. Natl. Acad. Sci. USA 111(33):E3395-404.
Nakagawa S. H., & Tager H. S. 1989. Perturbation of insulin-receptor interactions by intramolecular hormone cross-linking. Analysis of relative movement among residues A1, B1, and B29. J. Biol. Chem. 264(1):272-9.
Phillips, N. B., Whittaker, J., Ismail-Beigi, F., & Weiss, M. A. (2012) Insulin fibrillation and protein design: topological resistance of single-chain analogues to thermal degradation with application to a pump reservoir. J. Diabetes Sci. Technol. 6, 277-288.
Rohl, C. A., Chakrabartty, A., & Baldwin, R. L. 1997. Protein Soc. 5:2623-2637.
Vinther, T. N., Pettersson, I., Huus, K., Schlein, M., Steensgaard, D. B., Sorensen, A., Jensen, K. J., Kjeldsen T., and Hubalek, F. 2015. Additional disulfide bonds in insulin: Prediction, recombinant expression, receptor binding affinity, and stability. Protein Sci. 24:779-88.

Vinther, T. N., Norrman, M., Ribel, U., Huus, K., Schlein, M., Steensgaard, D. B., Pedersen, T. A., Pettersson, I., Ludvigsen, S., Kjeldsen, T., Jensen, K. J., and Hubalek, F. 2013. Insulin analog with additional disulfide bond has increased stability and preserved activity. Protein Sci. 22:296-305.

Wang, Z. X. 1995. An exact mathematical expression for describing competitive biding of two different ligands to a protein molecule FEBS Lett. 360: 111-114.

Whittaker, J., and Whittaker, L. 2005. Characterization of the functional insulin binding epitopes of the full-length insulin receptor. J. Biol. Chem. 280: 20932-20936.
Yutani, K., Ogasahara, K., Sugino, Y. 1985. Effect of amino-acid substitutions on conformational stability of a protein. Adv Biophys. 20:13-29.

What is claimed is:

1. A single-chain insulin analogue comprising a polypeptide having the sequence of the B-chain of insulin, a polypeptide comprising the sequence of the A-chain of insulin and a C-domain connecting polypeptide between the B-chain polypeptide sequence and the A-chain polypeptide sequence, wherein the A-chain polypeptide sequence comprises a substitution at at least one position corresponding to position A8 and position A14 of insulin, and wherein the C-domain comprises residues selected from the group consisting of residues 31-36 of SEQ ID NO:5, residues 31-37 of SEQ ID NO: 8, residues 31-38 of SEQ ID NO: 9, and residues 31-39 of SEQ ID NO: 10.

2. The single-chain insulin analogue of claim 1, comprising a substitution at position A14 of any amino acid other than Tyr and Pro.

3. The single-chain insulin analogue of claim 2, wherein the substitution at position A14 is Glu.

4. A single-chain insulin analogue comprising any one of SEQ ID NOs: 6-10.

5. The single-chain insulin analogue according to claim 4, comprising SEQ ID NO: 6.

6. The single-chain insulin analogue according to claim 4, comprising SEQ ID NO: 7.

7. The single-chain insulin analogue according to claim 4, comprising SEQ ID NO: 8.

8. The single-chain insulin analogue according to claim 4, comprising SEQ ID NO: 9.

9. The single-chain insulin analogue according to claim 4, comprising SEQ ID NO: 10.

10. A method for lowering the blood sugar level of a patient in need thereof, the method comprising, subcutaneously administering a pharmaceutical composition to the patient, wherein the pharmaceutical composition comprises a single-chain insulin comprising a polypeptide having the sequence of the B-chain of insulin, a polypeptide comprising the sequence of the A-chain of insulin and a C-domain connecting polypeptide between the B-chain polypeptide sequence and the A-chain polypeptide sequence, wherein the A-chain polypeptide sequence comprises a substitution at at least one position corresponding to position A8 and position A14 of insulin, and wherein the C-domain comprises residues selected from the group consisting of residues 31-36 of SEQ ID NO:5, residues 31-37 of SEQ ID NO: 8, residues 31-38 of SEQ ID NO: 9, and residues 31-39 of SEQ ID NO: 10.

11. The method of claim 10, wherein the insulin analogue comprises a substitution at position A14 of any amino acid other than Tyr and Pro.

12. The method of claim 11, wherein the substitution at position A14 is Glu.

13. A method of treating a patient comprising administering a physiologically effective amount of a single-chain insulin analogue or a physiologically acceptable salt thereof to the patient, wherein the single-chain insulin analogue comprises the single-chain insulin analogue of claim 4.

14. The method of claim 13, wherein the single-chain insulin analogue comprises SEQ ID NO: 6.

15. The method of claim 13, wherein the single-chain insulin analogue comprises SEQ ID NO: 7.

16. The method of claim 13, wherein the single-chain insulin analogue comprises SEQ ID NO: 8.

17. The method of claim 13, wherein the single-chain insulin analogue comprises SEQ ID NO: 9.

18. The method of claim 13, wherein the single-chain insulin analogue comprises SEQ ID NO: 10.

19. (canceled)

20. (canceled)

21. (canceled)

22. (canceled)


SEQUENCE LISTING (11 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 57 aa, Artificial Sequence
  synthetic construct
FVNQHLCGSHLVEALYLVCGERGFFYTDPTEEGPRRGIVEQCCHSICSLEQLENYCN

SEQ ID NO: 5 - protein, 57 aa, Artificial Sequence
  synthetic construct
  Misc Features (28)..(28): X is any amino acid
  Misc Features (28)..(29): One or both amino acids is an amino acid other than Pro
  Misc Features (29)..(29): X is any amino acid
  Misc Features (44)..(44): X is Thr, His, Ser, Glu or Ala
  Misc Features (50)..(50): X is any amino acid
FVNQHLCGSHLVEALYLVCGERGFFYTXXTEEGPRRGIVEQCCXSICSLXQLENYCN

SEQ ID NO: 6 - protein, 57 aa, Artificial Sequence
  synthetic construct
  Misc Features (31)..(31): X is Glu or Ala
  Misc Features (32)..(32): X is Glu or Ala
  Misc Features (33)..(33): X is Gly or Ala
  Misc Features (34)..(34): X is Pro or Ala
  Misc Features (35)..(35): X is Arg or Ala
  Misc Features (36)..(36): X is Arg or Ala
  Misc Features (44)..(44): X is Thr, His, Ser, Glu or Ala
  Misc Features (50)..(50): X is any amino acid other than Pro
FVNQHLCGSHLVEALYLVCGERGFFYTDPTXXXXXXGIVEQCCXSICSLXQLENYCN

SEQ ID NO: 7 - protein, 57 aa, Artificial Sequence; identical to SEQ ID NO: 6
  synthetic construct
  Misc Features (31)..(36): X is 4 to 5 amino acids
  Misc Features (31)..(31): X is Glu or Ala or is deleted
  Misc Features (32)..(32): X is Glu or Ala or is deleted
  Misc Features (33)..(33): X is Gly or Ala or is deleted
  Misc Features (34)..(34): X is Pro or Ala or is deleted
  Misc Features (35)..(35): X is Arg or Ala or is deleted
  Misc Features (36)..(36): X is Arg or Ala or is deleted
  misc_feature (44)..(44): Xaa can be any naturally occurring amino acid
  Misc Features (50)..(50): X is any amino acid other than Pro

SEQ ID NO: 8 - protein, 58 aa, Artificial Sequence
  synthetic construct
FVNQHLCGSHLVEALYLVCGERGFFYTDPTEEGAPRRGIVEQCCHSICSLEQLENYCN

SEQ ID NO: 9 - protein, 59 aa, Artificial Sequence
  synthetic construct
FVNQHLCGSHLVEALYLVCGERGFFYTDPTEEGAAPRRGIVEQCCHSICSLEQLENYCN

SEQ ID NO: 10 - protein, 60 aa, Artificial Sequence
  synthetic construct
FVNQHLCGSHLVEALYLVCGERGFFYTDPTEEGAAAPRRGIVEQCCHSICSLEQLENYCN

SEQ ID NO: 11 - protein, 30 aa, Artificial Sequence
  synthetic construct
FVNQHLCGSHLVEALYLVCGERGFFYTKPT